(19) United States
(12) Patent Application Publication
(10) Pub. No.: US 2022/0087603 A1
(43) Pub. Date: Mar. 24, 2022

(54) System and Method for Subject Identification of Biosensor Data

(71) Applicant: Bob M. Lansdorp, Santa Barbara, CA (US)

(72) Inventor: Bob M. Lansdorp, Santa Barbara, CA (US)

(21) Appl. No.: 17/448,682

(22) Filed: Sep. 23, 2021

Related U.S. Application Data
(60) Provisional application No. 62/707,000, filed on Sep. 23, 2020.

(51) Int. Cl. A61B 5/00; G06K 9/00; G06K 9/62; G06T 7/00; H04M 1/72409; A61B 5/117

(52) U.S. Cl. CPC A61B 5/4845; G06K 9/00362; G06K 9/6215; G06T 7/0002; H04M 1/72409; A61B 5/681; A61B 5/0013; A61B 5/002; A61B 5/117; A61B 5/746; A61B 5/6844

(57) ABSTRACT

Verification of biosensor data by determining a tamper event. The tamper event can be detected as any event which attempts to spoof the system by obtaining incorrect or improper readings. One verification obtains a known image of the user and compares that with an image of the user which is taken at a time that the biosensor is placed in contact with the user skin. A similarity score between the known image and the taken image can be obtained, and a tamper alert generated when the similarity score is not sufficiently high to indicate a match.

### BACKGROUND

### Field of the Invention

The present invention is related to the reliable detection of biosensor data from a human subject.

### Description of Related Art

The reliable detection of alcohol consumption is important in family law disputes, where consumption of alcohol can impact child-custody. Reliable detection of alcohol consumption can also be important in professional settings, where abstinence during work can be a condition of employment, for example for airline pilots.

One example of a previously developed transdermal alcohol sensor technology is shown in U.S. Pat. No. 9,489,487. This is also called the SCRAM transdermal alcohol sensor by Alcohol Monitoring Systems, which provides a means of detection of alcohol consumption by means of a platinum fuel-cell sensor that is attached to an ankle-bracelet, and transmits data to a server via a modem. Although the built-in infrared diode allows the SCRAM device to measure skin proximity, the system is bulky, and the ankle-bracelet cannot be removed, which causes skin irritation and inconvenience for the wearer.

Therefore, what is desired is a method that enables the system to be removed and re-attached, to facilitate swimming, scuba diving, and other water-related activities.

There exist numerous breathalyzer systems such as the Soberlink Breathalyzer (U.S. Pat. No. 9,228,997 B2), BACTrack breathalyzer by KHN solutions (USD 727,764 S1), SCRAM breathalyzer (U.S. Pat. No. 9,829,480), that enable readings to be taken by means of a breath sample and which also enable simultaneous subject identification by means of visual recording of the wearer. The measurements can verify that the breath readings correspond to the person that purports to be taking the readings. However, these systems are not continuous, and only give one data point per reading. The breathalyzers must be carried, which is a burden on the wearer. Furthermore, scheduling of multiple readings per day is an inconvenience to the wearer.

Therefore, what is desired is a means of continuous alcohol consumption measurement that alleviates the burden of multiple check-ins per day, that combines with a means of subject identification.

Lansdorp (U.S. Pat. No. 9,855,000) describes a transdermal alcohol sensor that utilizes a disposable enzymatic sensor cartridge. Although the system is discreet and small in size as compared to the SCRAM ankle bracelet, and it does enable the measurement of alcohol consumption, it does not include a detailed tamper detection system.

Therefore, what is desired is to improve combine the novel transdermal analyte sensing system with a new system of tamper detection.

### SUMMARY

This invention is a method for reliable detection of biosensor data such as alcohol consumption, by detecting a tamper event, and correlating that with the obtaining of the wearer's biosensor data.

In an embodiment, the tamper event can be a comparison of an image taken of the person wearing the biosensor with a known image of that person, along with determining timestamps of the biosensor data and the image taken data.

In another embodiment, the tamper event can be an image taken of an operative part of the biosensor, for example a cartridge of the biosensor. The image can be analyzed to determine if the cartridge was tampered with in any way.

### BRIEF DESCRIPTION OF THE DRAWINGS

FIG. 1 shows a schematic illustration of a wearable transdermal alcohol sensor, a smartphone with display, and the cloud; and

FIG. 2 shows a schematic illustration of a wearable transdermal alcohol sensor with integrated proximity sensor, a smartphone with display button to synchronize data;

FIGS. 3A-3D show a schematic of various stages of a smartphone app walk-through;

FIG. 4 shows a schematic of data flow to generate a tamper alert;

FIG. 5 shows a schematic of data flow to generate a tamper alert for a user whose image does not correspond with unknown likeness;

FIG. 6 shows a schematic of data flow to generate a tamper alert.

### DETAILED DESCRIPTION

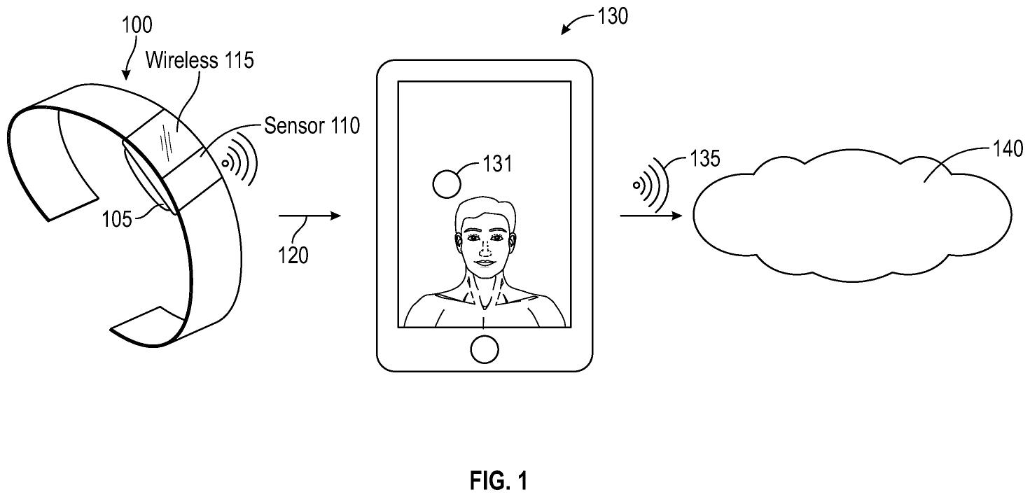

In an embodiment shown in FIG. 1, a transdermal alcohol sensor 110 and skin proximity sensor 105 are combined into a wearable device 100 that includes wireless communication capability 115 that transmits data 120 to a mobile phone 130 that has a built in video camera 131 and connects 135 to the cloud 140. The mobile phone 130 can be a smart phone. In alternative embodiments, this can alternatively connect to other portable computers such as tablets and the like. The wearable can store data temporarily on an onboard FLASH memory.

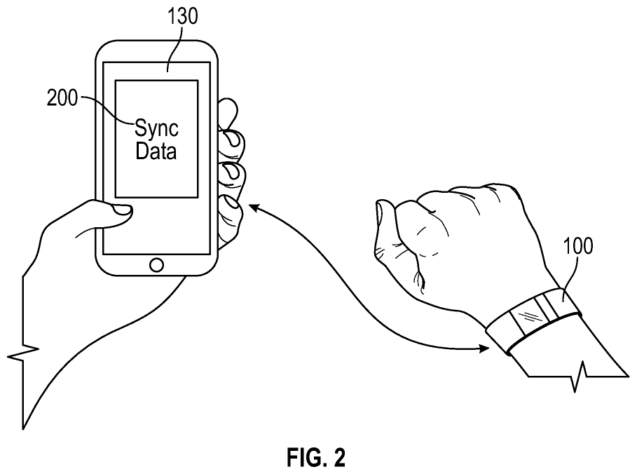

In FIG. 2, the smartphone 130 is shown initiating a connection with the wearable device 100 to synchronize data using a button 200 that appears on the display. For example, the button can read “Sync now”, displayed on the smartphone display. Initiating this button causes the Bluetooth connection between the wearable and the phone to be initiated and data is retrieved from memory on the wearable 100, sent over Bluetooth, and received on the smartphone 130.

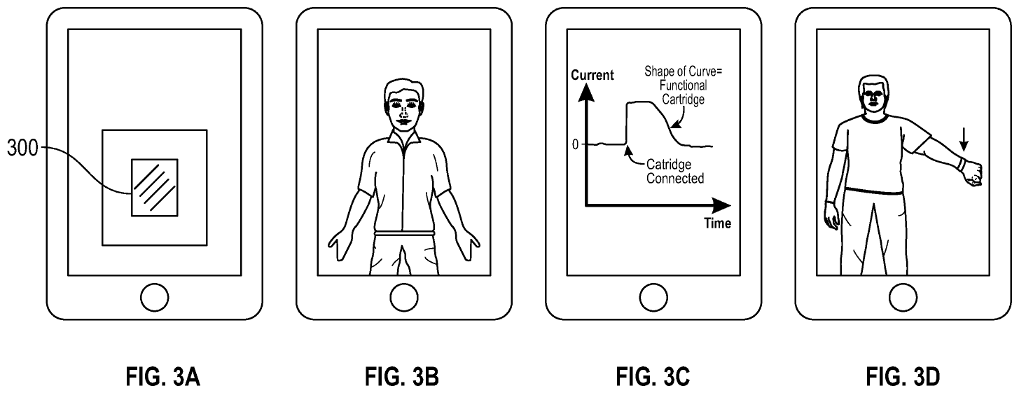

FIGS. 3A-3D illustrate a smartphone application walk-through, which depicts various features of determining data on the phone and also determining a tamper event. As used herein, the term “tamper event” refers to any action taken to attempt to spoof a biochemical or other measurement taken by the wearable.

First, at FIG. 3A, the camera 131 in the smartphone 130 is used to analyze a cartridge package 300 that the wearer is holding. The camera 131 takes an image, which can be a still image, or video, or any other kind of image. The term image is used herein to include both image and video, based on the convention that a video is a series of images. This image is analyzed to detect whether or not the cartridge package has been tampered with in any way, by analyzing using visual or machine vision inspection for tears in the packaging. The cartridge package may also have a tamper-resistant seal which can be analyzed using the image. Alternatively, the cartridge package may contain a temperature-sensitive indicator strip, that changes color if the temperature exceeds certain bounds. Thus, a cartridge packaging tamper event can be detected visually.

Next, at FIG. 3B, a video camera 131 built into the smartphone 130 records a video or image that is used to verify the identity of the person at FIG. 3B, by comparing the image that was taken to a likeness of that person. The likeness could be a previously taken photograph, video, Apple's Face ID, or other data familiar to those skilled in the art that can be used to determine similarity.

Next, at FIG. 3C, for an enzymatic transdermal alcohol sensor, a cartridge is inserted into the wristband. This results in an immediate change to the transdermal sensor readings and the skin proximity readings. Although the wearable is not yet on skin at this point, a change in capacitance from the no-connection state to cartridge-connected state results in a measurable change that can determine that a cartridge is correctly connected. When the cartridge is attached to the wristband housing, a potential is applied between the terminals, and the electrical current is measured that is proportional to electrochemical reduction or oxidation on the electrode surface. This measured electrochemical signal going to saturation for at least 10 seconds can verify that a new and functional cartridge (see Lansdorp et al 2019).

Next, at FIG. 3D, an image is taken, using the video camera 131 and the smart phone 130, of the wristband being placed on the wrist of the wearer.

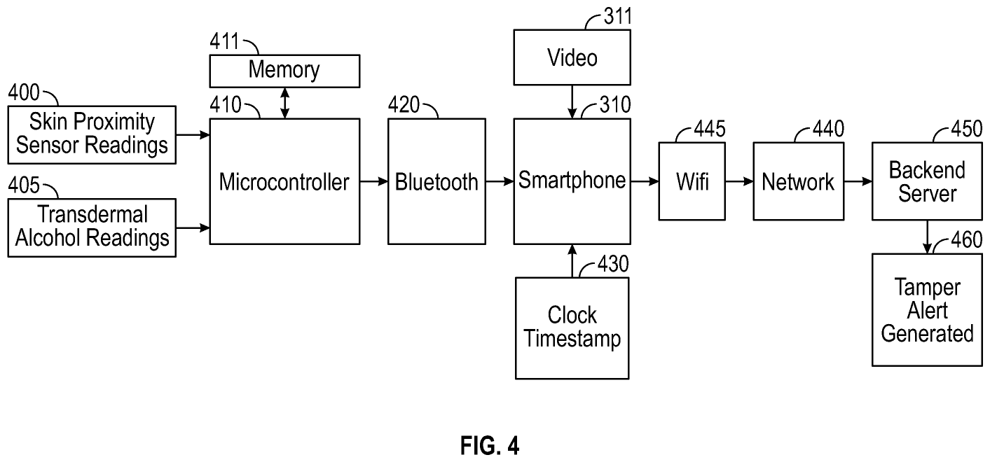

A schematic of the tamper alert generation system is shown in FIG. 4. Skin proximity sensor readings 400 and transdermal sensor alcohol readings 405 are sent to a microcontroller 410. The microcontroller may optionally store data on flash memory 411. Once the bluetooth connection has been established at 420 between the microcontroller 410 and the smartphone 310, data is retrieved from memory and is sent over bluetooth. The smartphone concurrently collects image from an onboard digital camera and timestamps from a clock 430. The smartphone can be an Apple iPhone or a Samsung phone running an Android Operating system, or other types familiar to those skilled in the art. The smartphone connects to a network 440, for example by means of wifi 445 or cellular data such as 4G or 5G. Data is routed through a network to a backend server 450, where raw data is processed. At the backend server 450, the data is analyzed to generate a tamper alert shown as 460. The tamper alert can result from any attempt to spoof the system.

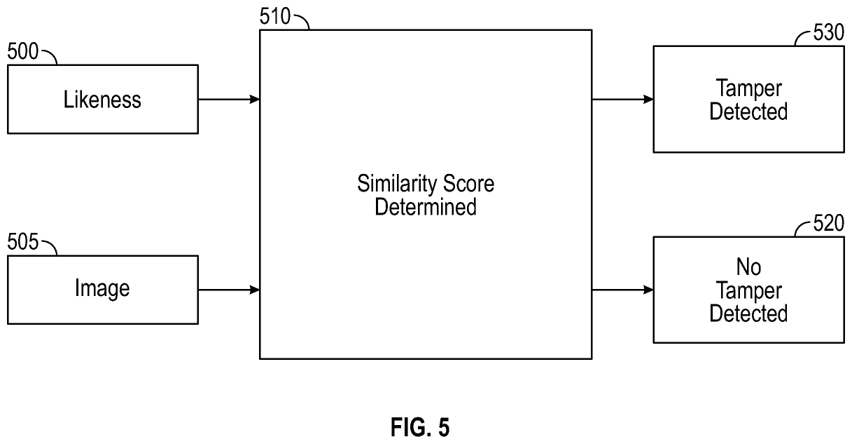

In FIG. 5, a schematic of data flow is shown. This data flow can be carried out by the back end server 450, or by some other computer. A known likeness 500 of an individual, for example a photograph of the individual's face, is used as a basis for comparison and can be a known likeness established during an initial operation or set up of the device. This likeness is compared to a image 505 of the individual, for example taken by a smartphone camera. The likeness and image are compared, to determine similarity at 510, to determine a similarity score. This can be performed using any of the many means of comparing similarity, such as facial recognition programs such as Deep Vision AI, SenseTime, Amazon Rekognition, FaceFirst, Trueface, Face++, Kairos, Cognitec, or others means familiar to those skilled in the art. The system determines the likeness and the image to be the same person at 520, if the score is greater than a specified amount. If the similarity score indicates that the likeness and the image are the same person, then this establishes that there is no tamper detection at 520. If the similarity score indicates the likeness and the image are not the same person, this is taken as an attempt to spoof the system, thus leading to a tamper event being established at 530.

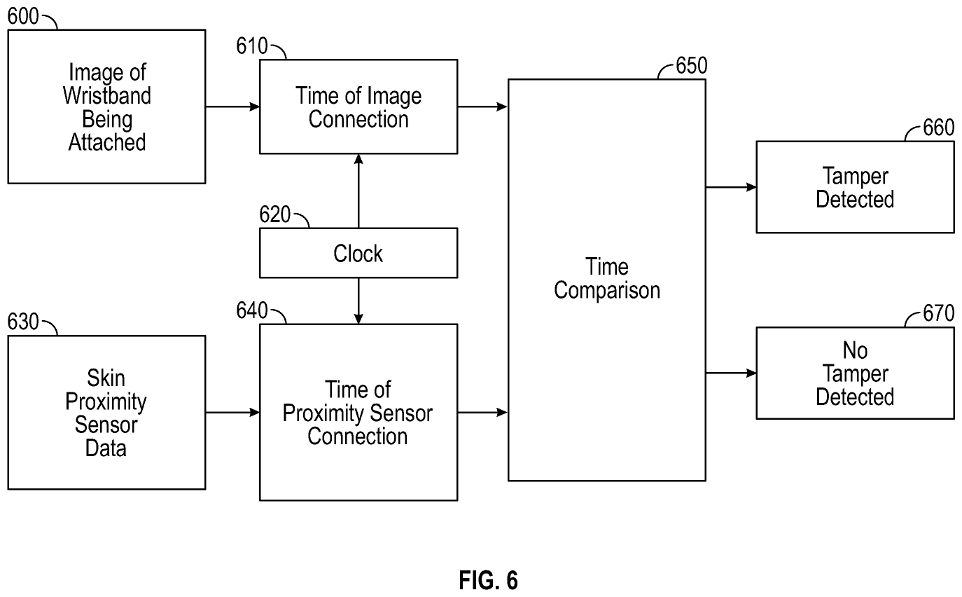

In FIG. 6, a schematic is shown of data flow leading to a tamper detection alert. A image is captured of a wearable sensor being attached to the individual wearing it. For example, a wristband is shown in the same frame as a given individual's wrist, and then the wristband is observed to be attached to the skin. The time that a wristband is attached to the skin is obtained at 610 by means of a clock 620 in the smartphone. Concurrent to the images of the wearable being attached, the sensor data 630 from that same wearable is sent to the smartphone via Bluetooth. The time at which the sensor is measured to be attached to the skin obtained at 640 is compared to the image as a time comparison at 650. If the time differs beyond a nominal threshold, for example two seconds, then a tamper alert is generated at 660, as it indicates a possible attempt to spoof the system using a different person to obtain the readings. If the time does not vary by this nominal threshold, then no tamper alert is detected at 670.

In another embodiment, a tamper alert that is generated if skin proximity signal falls below a certain threshold during normal wear, indicating that the device has been removed or is no longer operating correctly.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. For example, this could be applied to other kinds and form factors of sensors. Thus, the present invention is not intended to be limited to the embodiments shown herein but is to be accorded the widest scope consistent with the principles and novel features disclosed herein.

1. A system for detection of biosensor data comprising:
a biosensor, having a skin proximity sensor which creates skin proximity sensor data when in contact with skin;
an electronics system which wirelessly communicates said skin proximity sensor data; and
an application running on a wireless device, which receives said skin proximity sensor, and also includes a camera, and which sends both said skin proximity sensor data, and image from the camera, to a network based computer which operates to determine if a tamper event has occurred using the image from the camera.

2. The system as in claim 1, wherein the skin proximity sensor uses a cartridge, the data from the camera includes an image of an aspect of the cartridge, and the tamper event comprises tampering with the cartridge as determined from the image of the aspect of the cartridge.

3. The system as in claim 1, wherein the wireless device also obtains a time at which the image was obtained, and also uses the time at which the image was obtained to determine the tamper event.

4. The system as in claim 1, wherein the tamper event comprises an unauthorized person wearing the biosensor, determined by using the camera to obtain an image of the person wearing the biosensor and comparing the image of the person to a known image of the person, and detecting a tamper event when the image of the person does not match to the known image.

5. The system as in claim 1, wherein the tamper event is detected by detecting a first time of an image of a person wearing the biosensor, a second time of obtaining skin proximity sensor data, and comparing the first time to the second time to detect a tamper event when the first time differs by a specified amount from the second time.

6. The system as in claim one, wherein the tamper event includes detecting an unauthorized person wearing the sensor.

7. The system of claim 1, wherein the biosensor is a transdermal alcohol sensor.

8. The system of claim 1, wherein the skin proximity sensor is a capacitive sensor.

9. The system of claim 1, wherein the wireless communication uses a Bluetooth format.

10. A method for verifying detection of biosensor data from a wearer comprising:
obtaining a known image;
using a camera for obtaining an image of a wearer wearing a body-worn sensor;
determining an image time, indicating when the image of the wearer was obtained;
using a computer for comparing the image of the wearer to the known likeness, to determine a degree of similarity score between the image of the wearer and the known likeness;
issuing a tamper alert if the degree of similarity falls below a predetermined value;
obtaining skin proximity readings from the body-worn sensor;
obtaining a proximity connection time that corresponds to a time at which skin proximity sensor values change to greater than a predetermined threshold which indicates that the skin sensor is in contact with skin;
comparing the proximity connection time to the image time and establishing a tamper alert if a difference between the proximity connection time in the image time exceeds a threshold; and
establishing the skin proximity readings as being valid when no tamper alerts have been detected.

11. The method of claim 10, further comprising a tamper alert that is generated if skin proximity signal falls below a certain threshold during normal wear.

12. The method of claim 10, wherein the obtaining skin proximity readings comprises using a removable sensor cartridge to obtain skin proximity readings.

13. The method of claim 12, further comprising determining if the removable sensor cartridge has been tampered with as a tamper event.

14. The method as in claim 13, wherein the removable sensor cartridge is determined to have not been tampered with evaluating a temperature-indicator in the cartridge package.

15. The method of claim 5, further comprising using the sensor to obtain transdermal alcohol sensor signal readings.
